(19) United States
(12) Patent Application Publication
(10) Pub. No.: US 2021/0128070 A1
(43) Pub. Date: May 6, 2021

(54) METHOD AND APPARATUS FOR ANALYSING SIGNAL

(71) Applicant: LG ELECTRONICS INC., Seoul (KR)

(72) Inventor: Taehwan KIM, Seoul (KR)

(73) Assignee: LG ELECTRONICS INC., Seoul (KR)

(21) Appl. No.: 16/791,874

(22) Filed: Feb. 14, 2020

(30) Foreign Application Priority Data
Oct. 31, 2019 (KR) 10-2019-0137551

(51) Int. Cl. A61B 5/00; G06K 9/62; G06N 20/20; G06N 3/08

(52) U.S. Cl. CPC A61B 5/7242; G06K 9/6221; G06N 3/08; G06N 20/20; G06K 9/6232

(57) ABSTRACT

A method and apparatus for analysing a signal is disclosed. The method for analysing the signal includes collecting signals needed to learn signal analysis, clustering features of the signals on a feature space, generating a signal analysis model for each of clusters formed by the clustering, and integrating outputs of the signal analysis models generated for the clusters. According to the present disclosure, it is possible to robustly diagnose a testee based on biomedical signal analysis that uses an artificial intelligence (AI) model through a 5G network.

### CROSS-REFERENCE TO RELATED APPLICATION

Pursuant to 35 U.S.C. § 119(a), this application claims the benefit of earlier filing date and right of priority to Korean Patent Application No. 10-2019-0137551, filed on Oct. 31, 2019, the contents of which are all hereby incorporated by reference herein in their entirety.

### BACKGROUND

### 1. Technical Field

The present disclosure relates to a method and apparatus for analysing a signal, and more particularly, to a method and apparatus for diagnosing a state of a testee via biomedical signal analysis that is adaptive to changes in a situation and environment of the testee.

### 2. Description of Related Art

One related art, relating to an apparatus for predicting a state of a patient based on artificial intelligence, is disclosed in KR publication. This related art discloses predicting the state of a patient by learning biomedical signals collected from the patient after surgery, according to a type of the patient that is classified based on the biomedical signals collected from the patient before surgery. However, this related art does not disclose predicting the state of the patient based on biomedical signal analysis that changes according to a situation or environment of the patient.

Further, another related art, relating to a system for diagnosing a disease based on artificial intelligence by using a linkage of biomedical information and image information, is disclosed in KR publication. This related art merely discloses a system for determining the disease by using thermal images and biomedical signals, and then updating a database to include feedback regarding the presented disease from an administrator. However, this related art does not disclose predicting the state of the patient based on biomedical signal analysis that changes according to a situation or environment of the patient.

### SUMMARY OF THE INVENTION

The present disclosure is directed to addressing a disadvantage associated with some related art in which, since there is a difference between a learning environment and a testing environment, testing results fall short of learning results in using an artificial intelligence model for signal analysis.

In addition, the present disclosure is further directed to addressing a disadvantage associated with some related art in which, since an artificial intelligence model for signal analysis is not adaptive to changes in a testing environment, results that fall short of expected results are outputted.

The present disclosure is not limited to what has been disclosed hereinabove. Other objectives and advantages of the present disclosure which are not mentioned will be more clearly understood from the following embodiments. In addition, it will be understood that the objectives and the advantages of the present disclosure can be realized by the means recited in claims and a combination thereof.

A method for analysing a signal according to one embodiment of the present disclosure may include: collecting signals needed to learn signal analysis; clustering features of the signals on a feature space; generating a signal analysis model for each of clusters formed by the clustering; and integrating outputs of the signal analysis models generated for the clusters.

In addition, the collecting the signals may include collecting biomedical signals generated from a testee to predict a state of the testee.

In addition, the collecting the biomedical signals may include, under a current situation or environment of the testee: receiving raw biomedical signals from the testee; and processing the raw biomedical signals.

In addition, the biomedical signals may include at least one of a movement signal, a breathing signal, or a heartbeat signal of the testee.

In addition, the clustering may include extracting features from the signals that are in time series; and clustering the features on the feature space.

In addition, the clustering may include determining probabilities for the clusters formed by the features extracted from the signals.

In addition, the generating the signal analysis model may include generating the signal analysis model that is adaptive to a situation or environment of a signal source by using an ensemble technique.

In addition, the integrating the outputs of the signal analysis models may include determining, based on probability values for the clusters formed by the features extracted from the signals, an ensemble weight to be applied to each of the clusters.

The method for analysing the signal may further include predicting a state of the signal source based on the result of integrating the outputs.

In addition, the predicting the state of the signal source may include predicting the state of the signal source by using the signal analysis model to which the ensemble weight is applied.

In addition, the determining the ensemble weight may include setting the ensemble weight as a linear or nonlinear function of probability values for the clusters.

An apparatus for analysing a signal according to one embodiment of the present disclosure may include: a processor configured to process collected signals, extract features from the signals, generate a signal analysis ensemble model to be trained via learning of the features, and control an output of the signal analysis ensemble model; a learning processor configured to train the signal analysis ensemble model via learning; and a signal analysis ensemble model configured to obtain a trained deep neural network assemble for predicting, via training, a state of a signal source from which the signals originated. Further, the signal analysis ensemble model may be configured to include a plurality of signal analysis models corresponding to clusters formed by clustering of the features, and integrate and output outputs of the plurality of signal analysis models generated for the clusters.

In addition, the signal may include biomedical signals generated from a testee as the signal source.

In addition, the processor may be configured to process the biomedical signals collected under a current situation or environment of the testee.

In addition, the processor may be configured to extract the features from the biomedical signals of the testee and cluster the extracted features on a feature space by using a clustering model.

In addition, the processor may be configured to determine probabilities for the clusters formed by the features extracted from the signals.

In addition, each of the plurality of signal analysis models may correspond to a biomedical signal analysis model that is adaptive to the situation or environment of the testee as the signal source, by using an ensemble technique.

In addition, the processor may be configured to determine, based on probability values for the clusters formed by the features extracted from the biomedical signals of the testee, an ensemble weight to be applied to each of the clusters.

In addition, the processor may be configured to predict a state of the testee by using the signal analysis ensemble model to which the ensemble weight is applied.

In addition, the processor may be configured to set the ensemble weight as a linear or nonlinear function of probability values for the clusters.

According to the present disclosure, it is possible to robustly diagnose the testee based on signal analysis via learning in a testing environment.

In addition, according to the present disclosure, it is possible to reduce errors in predicting the state of the testee by covering the range of biomedical signals collected from the testee according to changes in an environment.

### BRIEF DESCRIPTION OF THE DRAWINGS

FIG. 1 is an exemplary diagram of signal analysis, according to one embodiment of the present disclosure.

FIG. 2 is an exemplary diagram of a network environment to which an apparatus for analysing a signal is connected, according to one embodiment of the present disclosure.

FIG. 3 is a block diagram of an apparatus for analysing a signal, according to one embodiment of the present disclosure.

FIG. 4 is a block diagram of a learning device, according to one embodiment of the present disclosure.

FIG. 5 is a flowchart of a method for analysing a signal, according to one embodiment of the present disclosure.

FIG. 6 is an exemplary diagram of a method for analysing a signal, according to one embodiment of the present disclosure.

FIG. 7 is an exemplary diagram of a signal process, according to one embodiment of the present disclosure.

FIG. 8 is an exemplary diagram of a signal process, according to another embodiment of the present disclosure.

### DETAILED DESCRIPTION

Hereinafter, embodiments of the present disclosure will be described in detail with reference to the accompanying drawings. Like reference numerals refer to the like elements throughout and a duplicate description thereof is omitted. In the following description, the suffixes “module” and “unit” that are mentioned with respect to the elements used in the present description are merely used individually or in combination for the purpose of simplifying the description of the present disclosure, and therefore, the suffix itself will not be used to differentiate the significance or function or the corresponding term. In addition, in the following description of the embodiments disclosed in this specification, the detailed description of related known technology will be omitted based on the fact that it may obscure the subject matter of the embodiments according to the present disclosure. The accompanying drawings are used to help easily explain various technical features and it should be understood that the embodiments presented herein are not limited by the accompanying drawings. As such, the present disclosure should be construed to extend to any alterations, equivalents and substitutes in addition to those which are particularly set out in the accompanying drawings.

Although the terms first, second, and the like, may be used herein to describe various elements, these elements should not be limited by these terms. These terms are generally only used to distinguish one element from another.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present.

Biomedical signals are signals used to extract information from a biological system. A human body consists of several systems, including, for example, the nervous system, the cardiovascular system, and the musculoskeletal system. Each of the systems consists of several subsystems capable of performing many physiological processes.

The physiological processes are complex phenomena that involve stimulations and controls of nerves or hormones, inputs and outputs in the form of physical quantities, neurotransmitters or information, and mechanical, electrical or biomechanical activities.

Most physiological processes involve signals that reflect their properties and activities. These signals correspond to various forms of biomedical signals, including, for example, chemical signals in the form of hormones and neurotransmitters, electrical signals in the form of potentials or currents, and physical signals in the form of pressures or temperatures.

Bioelectric signals correspond to signals in the form of currents or voltages generated by nerve cells or muscle cells. Representative bioelectrical signals are, for example, electrocardiogram, electromyogram, and electroencephalogram signals, which are most widely used for diagnosis. The present disclosure is directed to providing a method for analysing a signal based on the bioelectrical signals, and an apparatus for analysing a signal using the same.

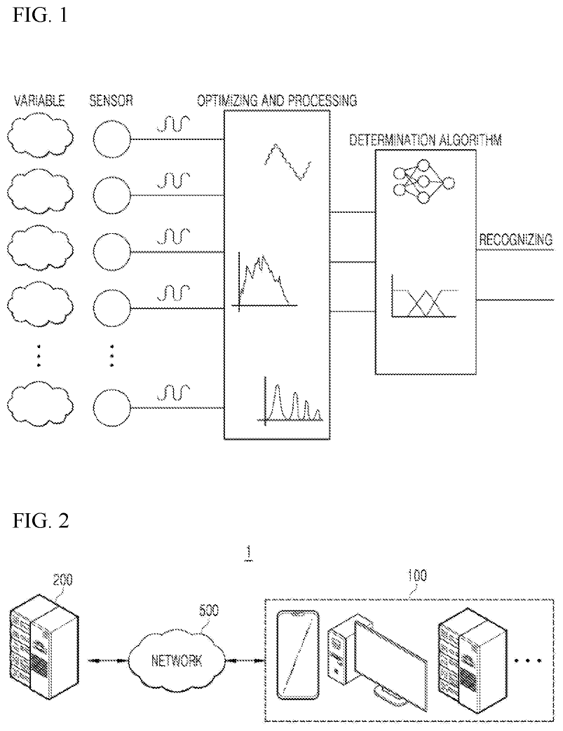

FIG. 1 is an exemplary diagram of signal analysis, according to one embodiment of the present disclosure.

Referring to FIG. 1, biomedical signals generated in a human body correspond to a set of variables that change in time series. In addition, sensors may collect these variables.

The sensors for collecting the biomedical signals are classified into contact type sensors or non-contact type sensors. The contact type sensors may include electrodes contacting the human body, and collect the bioelectrical signals via the electrodes. The non-contact type sensors may include a Doppler radar-based sensor and an RF resonance-based sensor.

In the signal analysis according to one embodiment of the present disclosure, the contact type sensors or the non-contact type sensors may be used, and the sensors may collect composite signals including various signals, for example, a heartbeat signal, a breathing signal, and a GSR signal.

The biomedical signals may be collected in the form of analog signals by the sensors, and converted into the form of digital signals during optimizing and processing processes. The optimizing and processing processes may include an amplification process, an ATD process, a noise removal process, and a signal decomposition process.

The apparatus for analysing the signal according to one embodiment of the present disclosure may analyse the biomedical signals via various determination algorithms by using data stored in the form of digital signals, and recognize the state of the testee based on a result of analysing the biomedical signals.

FIG. 2 is an exemplary diagram of a network environment to which an apparatus for analysing a signal is connected, according to one embodiment of the present disclosure.

Referring to FIG. 2, a network environment 1 is illustrated, including a terminal, a desktop computer, and a server computer, which correspond to the apparatus 100 for analysing the signal; a learning device 200; and a network 500 for communicatively connecting the foregoing components with each other, according to one embodiment of the present disclosure.

The apparatus 100 according to one embodiment of the present disclosure may be represented as an apparatus, such as the terminal, the desktop computer, or the server computer, according to the form of implementation, but is not limited to that illustrated in FIG. 2.

The apparatus 100 may use the learning device 100 during a signal analysis process. That is, the apparatus 100 may use artificial intelligence models, for example, deep neural networks, that have been trained by the learning device 200 and then stored therein. In addition, the apparatus 100 may analyse biomedical signals by using artificial intelligence models that have been downloaded and stored in the apparatus 100 and then trained by the learning device 200. Artificial intelligence will be described in detail below.

The learning device 200 may train and evaluate, via learning, the artificial intelligence models, for example, various neural networks, which are used to analyse the signal according to one embodiment of the present disclosure. The completed artificial intelligence models, having been evaluated, may be used by the apparatus 100 while being stored in the learning device 200 or the apparatus 100. The learning device 200 will be described in detail below.

The network 500 may be a wired or wireless network, such as a local area network (LAN), a wide area network (WAN), the Internet, the Intranet, and the Extranet. The network may also be a mobile network, such as a cellular, 3G, 4G, LTE, 5G, or WiFi network, an ad hoc network, and any suitable communication network including combinations thereof.

The network 500 may include connections between network components, such as a hub, a bridge, a router, a switch, and a gateway. The network 500 may include one or more connected networks, for example, a multi-network environment, including a public network such as the Internet and a private network such as a secure enterprise private network. Access to the network 500 may be provided through one or more wired or wireless access networks.

The apparatus 100 may transmit/receive data to/from the learning device 200 through a 5G network. Specifically, the apparatus 100 that is implemented as the terminal, may perform data communication with the learning device 200 by using at least one of an enhanced mobile broadband (eMBB) service, an ultra-reliable and low latency communications (URLLC) service, or a massive machine-type communications (mMTC) service through the 5G network.

The eMBB service is a mobile broadband service, and provides, for example, multimedia contents and wireless data access. In addition, improved mobile services such as hotspots and broadband coverage for accommodating the rapidly increasing mobile traffic may be provided by the eMBB service. Through hotspots, high-volume traffic may be accommodated in an area where user mobility is low and user density is high. Through the broadband coverage, a wide-range and stable wireless environment and user mobility may be guaranteed.

The URLLC service defines requirements that are far more stringent than existing LTE in terms of reliability and transmission delay of data transmission and reception, and corresponds to a 5G service for, for example, production process automation in industrial fields, telemedicine, remote surgery, transportation, and safety.

The mMTC service is a transmission delay-insensitive service that requires transmission of a relatively small amount of data. The mMTC service enables a much larger number of terminals such as sensors, than general mobile cellular phones, to be simultaneously connected to a wireless access network. In this case, a price of a communication module in the terminal should be low, and an improved technology to increase power efficiency and save power is required to enable its operation for several years without replacing or recharging a battery.

Artificial intelligence (AI) is an area of computer engineering science and information technology that studies methods to make computers mimic intelligent human behaviors such as reasoning, learning, self-improving, and the like.

In addition, artificial intelligence does not exist on its own, but is rather directly or indirectly related to a number of other fields in computer science. In recent years, there have been numerous attempts to introduce an element of AI into various fields of information technology to solve problems in the respective fields.

Machine learning is an area of artificial intelligence that includes the field of study that gives computers the capability to learn without being explicitly programmed.

More specifically, machine learning is a technology that investigates and builds systems, and algorithms for such systems, which are capable of learning, making predictions, and enhancing their own performance on the basis of experiential data. Machine learning algorithms, rather than only executing rigidly set static program commands, may be used to take an approach that builds models for deriving predictions and decisions from inputted data.

Numerous machine learning algorithms have been developed for data classification in machine learning. Representative examples of such machine learning algorithms for data classification include a decision tree, a Bayesian network, a support vector machine (SVM), an artificial neural network (ANN), and so forth.

Decision tree refers to an analysis method that uses a tree-like graph or model of decision rules to perform classification and prediction.

Bayesian network may include a model that represents the probabilistic relationship (conditional independence) among a set of variables. The Bayesian network may be appropriate for data mining via unsupervised learning.

SVM may include a supervised learning model for pattern detection and data analysis, heavily used in classification and regression analysis.

ANN is a data processing system modelled after the mechanism of biological neurons and interneuron connections, in which a number of neurons, referred to as nodes or processing elements, are interconnected in layers.

ANNs are models used in machine learning and may include statistical learning algorithms conceived from biological neural networks (particularly of the brain in the central nervous system of an animal) in machine learning and cognitive science.

ANNs may refer generally to models that have artificial neurons (nodes) forming a network through synaptic interconnections, and acquires problem-solving capability as the strengths of synaptic interconnections are adjusted throughout training.

The terms ‘artificial neural network’ and ‘neural network’ may be used interchangeably herein.

The ANN may include a number of layers, each including a number of neurons. In addition, the ANN may include synapses that connect the neurons to one another.

The ANN may be defined by the following three factors: (1) a connection pattern between neurons on different layers; (2) a learning process that updates synaptic weights; and (3) an activation function generating an output value from a weighted sum of inputs received from a lower layer.

The ANNs include, but are not limited to, network models such as a deep neural network (DNN), a recurrent neural network (RNN), a bidirectional recurrent deep neural network (BRDNN), a multilayer perception (MLP), and a convolutional neural network (CNN).

An ANN may be classified as a single-layer neural network or a multi-layer neural network, based on the number of layers therein.

In general, the single-layer neural network may include an input layer and an output layer.

In general, the multi-layer neural network may include an input layer, one or more hidden layers, and an output layer.

The input layer receives data from an external source, and the number of neurons in the input layer is identical to the number of input variables. The hidden layer is located between the input layer and the output layer, and receives signals from the input layer, extracts features, and feeds the extracted features to the output layer. The output layer receives a signal from the hidden layer and outputs an output value based on the received signal. Input signals between the neurons are summed together after being multiplied by corresponding connection strengths (synaptic weights), and the neuron can be activated and output an output value obtained through an activation function, based on this sum exceeding a threshold value of a corresponding neuron.

Meanwhile, the deep neural network including a plurality of hidden layers between the input layer and the output layer can be a representative artificial neural network that implements deep learning, which is a type of machine learning technology.

The ANN may be trained by using training data. Here, the training may refer to the process of determining parameters of the artificial neural network by using the training data, to perform tasks such as classification, regression analysis, and clustering of inputted data. Such parameters of the artificial neural network may include synaptic weights and biases applied to neurons.

The artificial neural network trained using training data may classify or cluster inputted data according to a pattern within the inputted data.

Throughout the present specification, the artificial neural network trained using training data may be referred to as a trained model.

Hereinbelow, learning paradigms of the artificial neural network will be described in detail.

The learning paradigms, in which the artificial neural network operates, may be classified into supervised learning, unsupervised learning, semi-supervised learning, and reinforcement learning.

Supervised learning is a machine learning method that derives a single function from the training data.

Among the functions that may be thus derived, a function that outputs a continuous range of values may be referred to as a regressor, and a function that predicts and outputs the class of an input vector may be referred to as a classifier.

In supervised learning, the artificial neural network may be trained with training data that has been given a label.

Here, the label may refer to a target answer (or a result value) to be guessed by the artificial neural network, based on the training data inputted to the artificial neural network.

Throughout the present specification, the target answer (or a result value) to be guessed by the artificial neural network, based on the training data inputted may be referred to as a label or labeling data.

Throughout the present specification, assigning one or more labels to training data in order to train the artificial neural network may be referred to as labeling the training data with labeling data.

Training data and label corresponding to the training data together may form a single training set, and as such, they may be inputted to the artificial neural network as a training set.

The training data may exhibit a number of features, and the training data being labeled with the labels may be interpreted as the features exhibited by the training data being labeled with the labels. In such a case, the training data may represent the feature of the input object in the form of a vector.

Using training data and labeling data together, the artificial neural network may derive a correlation function between the training data and the labeling data. Then, through evaluation of the function derived from the artificial neural network, a parameter of the artificial neural network may be determined (optimized).

Unsupervised learning is a machine learning method that learns from training data that has not been given a label.

More specifically, unsupervised learning may be a training scheme that trains the artificial neural network to discover a pattern within given training data and perform classification by using the discovered pattern, rather than by using a correlation between given training data and labels corresponding to the given training data.

Examples of unsupervised learning include, but are not limited to, clustering and independent component analysis.

Examples of artificial neural networks using unsupervised learning include, but are not limited to, a generative adversarial network (GAN) and an autoencoder (AE).

A GAN is a machine learning method in which two different artificial intelligences, a generator and a discriminator, improve performance through competing with each other.

The generator may be a model generating new data that generates new data based on true data.

The discriminator may be a model recognizing patterns in data that determines whether inputted data is from the true data or from the new data generated by the generator.

Furthermore, the generator may receive and learn from data that has failed to fool the discriminator, while the discriminator may receive and learn from data that has succeeded in fooling the discriminator. Accordingly, the generator may evolve so as to fool the discriminator as effectively as possible, while the discriminator evolves so as to distinguish, as effectively as possible, between the true data and the data generated by the generator.

An auto-encoder (AE) is a neural network which aims to reconstruct its input as output.

More specifically, AE may include an input layer, at least one hidden layer, and an output layer.

Since the number of nodes in the hidden layer is smaller than the number of nodes in the input layer, the dimensionality of data is reduced, thus leading to data compression or encoding.

Furthermore, the data outputted from the hidden layer may be inputted to the output layer. Given that the number of nodes in the output layer is greater than the number of nodes in the hidden layer, the dimensionality of the data increases, thus leading to data decompression or decoding.

Furthermore, in the AE, the inputted data is represented as hidden layer data as interneuron connection strengths are adjusted through training. The fact that when representing information, the hidden layer is able to reconstruct the inputted data as output by using fewer neurons than the input layer may indicate that the hidden layer has discovered a hidden pattern in the inputted data and is using the discovered hidden pattern to represent the information.

Semi-supervised learning is a machine learning method that makes use of both labeled training data and unlabeled training data.

One semi-supervised learning technique involves reasoning the label of unlabeled training data, and then using this reasoned label for learning. This technique may be used advantageously when the cost associated with the labeling process is high.

Reinforcement learning may be based on a theory that given the condition under which a reinforcement learning agent can determine what action to choose at each time instance, the agent can find an optimal path to a solution solely based on experience without reference to data.

Reinforcement learning may be performed mainly through a Markov decision process (MDP).

Markov decision process consists of four stages: first, an agent is given a condition containing information required for performing a next action; second, how the agent behaves in the condition is defined; third, which actions the agent should choose to get rewards and which actions to choose to get penalties are defined; and fourth, the agent iterates until future reward is maximized, thereby deriving an optimal policy.

The artificial neural network is characterized by features of its model, the features including an activation function, a loss function or cost function, a learning algorithm, an optimization algorithm, and so forth. Also, the hyperparameters are set before learning, and model parameters can be set through learning to specify the architecture of the artificial neural network.

For instance, the structure of the artificial neural network may be determined by a number of factors, including the number of hidden layers, the number of hidden nodes included in each hidden layer, input feature vectors, target feature vectors, and so forth.

Hyperparameters may include various parameters which need to be initially set for learning, much like the initial values of model parameters. Also, the model parameters may include various parameters sought to be determined through learning.

For instance, the hyperparameters may include initial values of weights and biases between nodes, mini-batch size, iteration number, learning rate, and so forth. Furthermore, the model parameters may include a weight between nodes, a bias between nodes, and so forth.

Loss function may be used as an index (reference) in determining an optimal model parameter during the learning process of the artificial neural network. Learning in the artificial neural network involves a process of adjusting model parameters so as to reduce the loss function, and the purpose of learning may be to determine the model parameters that minimize the loss function.

The loss function typically uses means squared error (MSE) or cross entropy error (CEE), but the present disclosure is not limited thereto.

The cross entropy error may be used when a true label is one-hot encoded. One-hot encoding may include an encoding method in which among given neurons, only those corresponding to a target answer are given 1 as a true label value, while those neurons that do not correspond to the target answer are given 0 as a true label value.

In machine learning or deep learning, learning optimization algorithms may be deployed to minimize a cost function, and examples of such learning optimization algorithms include gradient descent (GD), stochastic gradient descent (SGD), momentum, Nesterov accelerate gradient (NAG), Adagrad, AdaDelta, RMSProp, Adam, and Nadam.

GD includes a method that adjusts model parameters in a direction that decreases the output of a cost function by using a current slope of the cost function.

The direction in which the model parameters are to be adjusted may be referred to as a step direction, and a size by which the model parameters are to be adjusted may be referred to as a step size.

Here, the step size may mean a learning rate.

GD obtains a slope of the cost function through use of partial differential equations, using each of model parameters, and updates the model parameters by adjusting the model parameters by a learning rate in the direction of the slope.

SGD may include a method that separates the training dataset into mini batches, and by performing gradient descent for each of these mini batches, increases the frequency of gradient descent.

Adagrad, AdaDelta, and RMSProp may include methods that increase optimization accuracy in SGD by adjusting the step size. Momentum and NAG in SGD may include a method that increases optimization accuracy by adjusting the step size. Adam may include a method that combines momentum and RMSProp and increases optimization accuracy in SGD by adjusting the step size and step direction. Nadam may include a method that combines NAG and RMSProp and increases optimization accuracy by adjusting the step size and step direction.

Learning rate and accuracy of an artificial neural network rely not only on the structure and learning optimization algorithms of the artificial neural network but also on the hyperparameters thereof. Therefore, in order to obtain a good trained model, it is important to choose a proper structure and learning algorithms for the artificial neural network, but also to choose proper hyperparameters.

In general, the artificial neural network is first trained by experimentally setting hyperparameters to various values, and based on the results of training, the hyperparameters can be set to optimal values that provide a stable learning rate and accuracy.

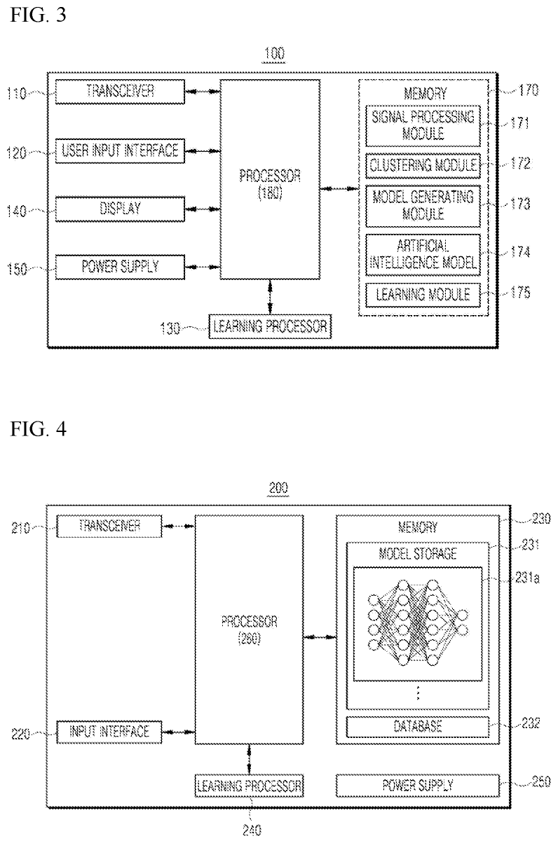

FIG. 3 is a block diagram of an apparatus for analysing a signal, according to one embodiment of the present disclosure.

Referring to FIG. 3, the apparatus 100 may be configured to include a transceiver 110, a user input interface 120, a learning processor 130, a display 140, a power supply 150, a memory 170, and processor 180.

The transceiver 110 may include a mobile communication module, a wireless Internet module, and a short-range communication module, which are used for wireless communication, and may also include a network card which is used for wired communication.

The user input interface 120 is for receiving information inputted from a user. Based on the information inputted via the user input interface 120, the processor 180 may control the apparatus 100 such that the operation of the apparatus 100 corresponds to the inputted information.

The learning processor 130 may train artificial intelligence models, for example, models composed of deep neural networks, via learning using training data.

The display 140 may display information processed by the apparatus 100. For example, the display 140 may display execution screen information of an application program executed in the apparatus 100, or user interface (UI) or graphic user interface (GUI) information according to the execution screen information.

Under the control of the processor 180, the power supply 190 is supplied with external power or internal power, and supplies power to each component included in the apparatus 100. The power supply 190 may include a battery, which may be an internal battery or a replaceable battery.

Various computer program modules may be loaded on the memory 170. The computer program modules loaded on the memory 170 may include, as application programs, a signal processing module 171, a clustering module 172, a model generating module 173, an artificial intelligence model 174, and a learning module 175, in addition to an operating system and a system program for managing a hardware.

Functions associated with the signal processing module 171, such as preprocessing of signals, for example, amplification, conversion to digital signals, noise removal, and signal decomposition of biomedical signals, may be performed via various computational functions of the processor 180.

Functions associated with the clustering module 172, such as extracting feature vectors from the biomedical signals and clustering the extracted feature vectors on a feature space, may be performed via various computational functions of the processor 180.

Functions associated with the model generating module 173, such as generating a signal analysis model for each of the clusters generated by the clustering, for example, a deep neural network for performing deep learning of signal analysis, and assigning weight to each signal analysis model, may be performed via various computational functions of the processor 180.

Functions associated with the artificial intelligence model 174, such as extracting features from signals and then clustering the extracted features, and analysing the signals, for example analysing time-series human body signals, may be performed via various computational functions of the processor 180.

A function associated with the learning module 175, such as retraining the pre-trained artificial intelligence models, for example, the deep neural networks, by using personal data of the user, may be performed via various computational functions of the processor 180 or the learning processor 130.

FIG. 4 is a block diagram of a learning device, according to one embodiment of the present disclosure.

The learning device 200 may be a device or server which is separately configured outside of the apparatus 100, and may perform the same function as the learning processor 130 of the apparatus 100.

That is, the learning device 200 may be configured to receive, classify, store, and output information to be used for data mining, data analysis, intelligent decision making, and machine learning algorithms. Here, the machine learning algorithm may include a deep learning algorithm.

The learning device 200 may communicate with at least one apparatus 100, and may analyse or learn data on behalf of the apparatus 100 or by assisting the apparatus 100 to derive a result. Here, assisting another apparatus may refer to a distribution of computing power by distributed processing.

The learning device 200 of the artificial neural network may be various devices for training the artificial neural network, may usually refer to a server, and may be referred to as, for example, a learning device or a learning server.

In particular, the learning device 200 may be implemented as not only a single server, but also, for example, a plurality sets of servers, a cloud server, or combinations thereof.

That is, a plurality of learning devices 200 may constitute a set of learning devices (or a cloud server), and at least one learning device 200 included in the set of learning devices may analyse or learn data by distributed processing to derive a result.

The learning device 200 may transmit a model trained via machine learning or deep learning to the apparatus 100 periodically or upon request.

Referring to FIG. 4, the learning device 200 may include, for example, a transceiver 210, an input interface 220, a memory 230, a learning processor 240, a power supply 250, and a processor 260.

The input interface 220 is a component corresponding to the user input interface 120 shown in FIG. 3, and may obtain data by receiving the data via the transceiver 210.

The input interface 220 may obtain, for example, training data for model training and input data for obtaining an output by using the trained model.

The input interface 220 may obtain unprocessed input data and in this case, the processor 260 may preprocess the obtained data to generate training data or preprocessed input data which may be inputted for model training.

Here, the preprocessing of the input data performed by the input interface 220 may refer to extracting an input feature from the input data.

The memory 230 is a component corresponding to the memory 170 shown in FIG. 3.

The memory 230 may include, for example, a model storage 231 and a database 232.

The model storage 231 stores a model (or an artificial neural network 231a) which is being trained or has been trained via the learning processor 240. Based on the model being updated via training, the model storage 231 stores an updated model.

Here, the model storage 231 may classify the trained model into a plurality of versions depending on, for example, training time or training progress, when necessary, and may store the classified trained model.

The artificial neural network 231a shown in FIG. 4, is provided as one example of the artificial neural network including a plurality of hidden layers. Accordingly, the artificial neural network according to one embodiment of the present disclosure is not limited thereto.

The artificial neural network 231a may be implemented as hardware, software, or a combination of hardware and software. Based on the artificial neural network 231a being partially or completely implemented as software, at least one command which constitutes the artificial neural network 231a, may be stored in the memory 230.

The database 232 stores, for example, input data obtained via the input interface 220, learning data (or training data) used to model training, and training history of the model.

The input data stored in the database 232 may be not only data suitably processed for model training but also unprocessed input data itself.

The learning processor 240 is a component corresponding to the learning processor 130 shown in FIG. 3.

The learning processor 240 may train the artificial neural network 231a by using training data or training set.

The learning processor 240 may train the artificial neural network 231a by immediately obtaining preprocessed data of the input data that the processor 260 obtains via the input interface 220, or may train the artificial neural network 231a by obtaining preprocessed input data stored in the database 232.

Specifically, the learning processor 240 may repeatedly train the artificial neural network 231a by using various training techniques described above to determine optimized model parameters of the artificial neural network 231a.

In this specification, the artificial neural network that is trained by using training data to determine the parameters may be referred to as a trained model.

In this case, the trained model may infer result values even while being loaded on the learning device 200 of the artificial neural network, and may be transmitted to another apparatus such as the apparatus 100 via the transceiver 210 to be loaded thereon.

In addition, based on the trained model being updated, the updated trained model may be transmitted to another apparatus such as the apparatus 100 via the transceiver 210 to be loaded thereon.

The power supply 250 is a component corresponding to the power supply 190 shown in FIG. 3.

Repeated description regarding components corresponding to each other will be omitted.

In addition, the learning device 200 may evaluate the artificial intelligence model 231a, and may update the artificial intelligence model 231a to have better performance even after the evaluation, and provide the updated artificial intelligence model 231a to the apparatus 100. Here, the apparatus 100 may perform a series of steps performed by the learning device 200, solely in a local area or together with the learning device 200 by communicating with the learning device 200. For example, by training the artificial intelligence model 174 in the local area with a personal pattern of a user via secondary training that uses personal data of the user, the apparatus 100 may update the artificial intelligence model 174 downloaded from the learning device 200.

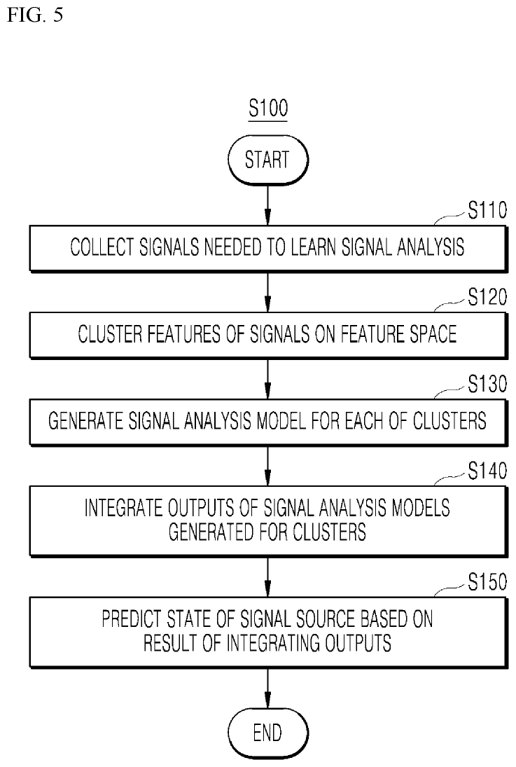

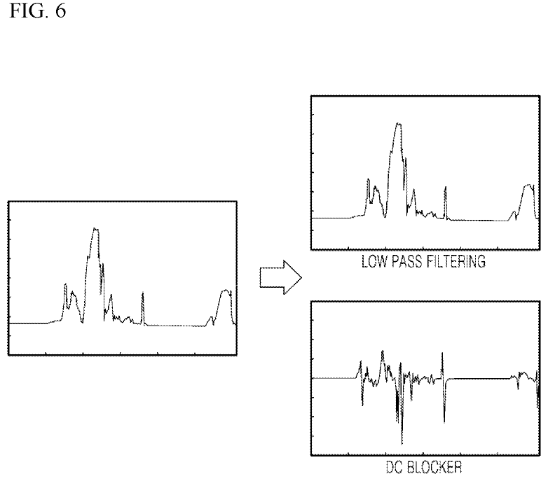

FIG. 5 is a flowchart of a method for analysing a signal, according to one embodiment of the present disclosure.

Referring to FIG. 5, the method (S100) for analysing the signal according to one embodiment of the present disclosure may include collecting signals (S110), clustering features of the signals (S120), generating signal analysis models (S130), integrating outputs of the signal analysis models generated for the clusters (S140), and predicting a state of a signal source based on the result of integrating the outputs (S150).

The processor 180, that is, the apparatus 100, may collect signals needed to learn signal analysis (S110).

Here, the collecting the signals (S110) may include collecting biomedical signals generated from a testee to predict a state of the testee. For example, the apparatus 100 may directly receive various monitoring signals from various sensors, or receive raw signal data transmitted from a biomedical signal monitoring system. The biomedical signal monitoring system may transmit signals detected from a human body by using various sensors, for example, the sensors in contact with the human body or the sensors not in contact with the human body, to the apparatus 100 in the form of data.

The collecting the biomedical signals may include, in receiving the biomedical signals under a current situation or environment of the testee, receiving raw biomedical signals from the testee (S111), and processing the raw biomedical signals (S112). The raw biomedical signals refer to unprocessed biomedical signals. The apparatus 100 may receive the raw biomedical signals from the testee, and convert the received raw biomedical signals into data for learning. This converting may include converting by using filters. The processor 180 may process the raw biomedical signals by using, for example, a low pass filter, a high pass filter, a band pass filter, a notch filter, and a DC blocker.

FIG. 6 is an exemplary diagram of a signal process, according to one embodiment of the present disclosure.

Referring to FIG. 6, signal waveforms which are outputted from a low pass filter and a DC blocker, respectively, after applying the low pass filter and the DC blocker to inputted raw biomedical signals, are illustrated.

In one embodiment of the present disclosure, the collected biomedical signals may include at least one of a movement signal, a breathing signal, or a heartbeat signal of the testee. That is, since the biomedical signals are composite biomedical signals that include one or more of the above signals, a process of decomposing the composite biomedical signals into the above signals is needed.

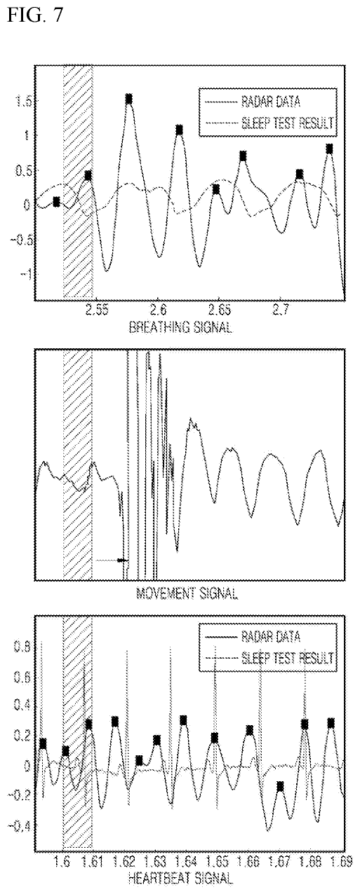

FIG. 7 is an exemplary diagram of a signal process, according to another embodiment of the present disclosure.

Referring to FIG. 7, waveforms of the breathing signal, the movement signal, and heartbeat signal that are decomposed from the composite signals are illustrated.

In order to analyse collected biomedical signals by using the artificial intelligence models, for example, the deep neural networks, it is necessary to train the signal analysis models with the biomedical signals.

The biomedical signals may include variables (hereinafter, referred to as composite factors) that are highly affected by a characteristic, state, and environment of the testee. Accordingly, due to the influence of the composite factors at the time of measuring the biomedical signals, unexpected results may occur in which the testing results using the artificial intelligence models for signal analysis fall short of the learning results. The unexpected results are due to the fact that the range in which the biomedical signals occur due to the composite factors may not be determined.

The apparatus 100 according to one embodiment of the present disclosure may be configured to include, as a signal analysis model capable of covering the entire range of the biomedical signals, a signal analysis ensemble model.

The process of designing the signal analysis ensemble model includes clustering the features of the signals, generating and training the signal analysis models for clusters generated by the clustering, and integrating the analysis results of each model.

First, the processor 180 may cluster the features of the signals on a feature space (S120). The clustering (S120) may include extracting features from the signals that are in time series (S121), and clustering the features on the feature space (S122). As a clustering algorithm for the features of the biomedical signals, for example, K-Means, Mean-Shift, DBSCAN, GMM, and Agglomerative Hierarchical may be used. In particular, based on the number of features of the biomedical signals being large, a feature dimension may be reduced, for example, by using a Principal Component Analysis (PCA) technique.

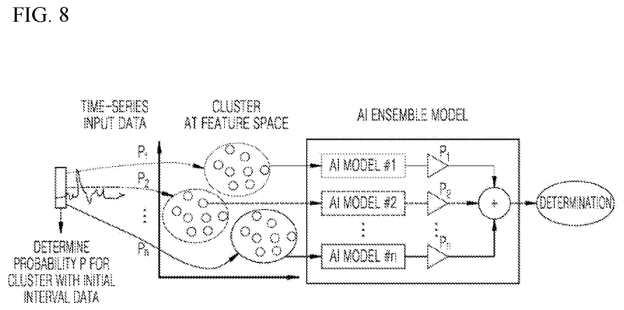

FIG. 8 is an exemplary diagram of the method for analysing the signal, according to one embodiment of the present disclosure.

Referring to FIG. 8, the feature space including the clusters formed by the features of the signals, and an artificial intelligence ensemble model generated for the clusters are illustrated.

Due to the distribution of the features of the biomedical signals included in a set of training data used for training the signal analysis models, 1 to N clusters may be formed in the feature space. In addition, for each of the formed clusters, the processor 180 may generate the signal analysis model for training with input data included in a corresponding cluster (S130).

The generating the signal analysis model may include generating a signal analysis model that is adaptive to a situation or environment of a signal source by using an ensemble technique. In addition, the apparatus 100 according to one embodiment of the present disclosure may analyse the biomedical signals by using the artificial intelligence ensemble model in signal analysis.

In this case, a clustering probability value P_{N} for each of the formed clusters may be computed.

The clustering may include determining probabilities for the clusters formed by the features extracted from the signals. The clustering probability value P_{N} does not need to be computed for all of the input signals, and may be computed for signals, for example, during an initial certain interval.

The processor 180 may integrate outputs of the signal analysis models generated for the clusters (S140). The features of the biomedical signals are divided into a plurality of clusters by clustering. Accordingly, a set of training data used to train the artificial intelligence models corresponding to the clusters includes the features of the signals suitable for a situation and environment of the testee. As such, in order to analyse the signals based on training the entire range of features of the signals, it is necessary to integrate the outputs of the artificial intelligence models.

The integrating the outputs of the signal analysis models may include determining, based on the probability value for each of the clusters formed by the features extracted from the signals, an ensemble weight to be applied to each of the clusters.

Referring to FIG. 8, signal analysis values outputted from 1 to n artificial intelligence models may be multiplied by the weights P₁ to P_{N}, respectively.

The processor may predict the state of the signal source based on the result of integrating the outputs (S150).

The predicting the state of the signal source may include predicting the state of the signal source by using the signal analysis models to which the ensemble weights are applied.

In addition, the ensemble weight may be determined by setting the ensemble weight as a linear or nonlinear function of the probability values for the clusters.

As described above, according to the embodiments of the present disclosure, it is possible to robustly diagnose the testee based on signal analysis via learning in a testing environment.

In addition, according to the present disclosure, it is possible to reduce errors in predicting the state of the testee by covering the range of biomedical signals collected from the testee according to changes in an environment.

Embodiments according to the present disclosure described above may be implemented in the form of computer programs that may be executed by various components on a computer, and such computer programs may be recorded on a computer-readable medium. Examples of the computer-readable medium include, but are not limited to: magnetic media such as hard disks, floppy disks, and magnetic tape; optical media such as CD-ROM disks and DVD-ROM disks; magneto-optical media such as floptical disks; and hardware devices that are specially configured to store and execute program codes, such as ROM, RAM, and flash memory devices.

Meanwhile, the computer programs may be those specially designed and constructed for the purposes of the present disclosure or they may be of the kind well known and available to those skilled in the computer software arts. Examples of computer programs may include both machine codes, such as produced by a compiler, and higher-level codes that may be executed by the computer using an interpreter.

As used in the present disclosure (especially in the appended claims), the terms “a/an” and “the” include both singular and plural references, unless the context clearly states otherwise. Also, it should be understood that any numerical range recited herein is intended to include all sub-ranges subsumed therein (unless expressly indicated otherwise) and therefore, the disclosed numeral ranges include every individual value between the minimum and maximum values of the numeral ranges.

Also, the order of individual steps in process claims of the present disclosure does not imply that the steps must be performed in this order; rather, the steps may be performed in any suitable order, unless expressly indicated otherwise. In other words, the present disclosure is not necessarily limited to the order in which the individual steps are recited. All examples described herein or the terms indicative thereof (“for example,” etc.) used herein are merely to describe the present disclosure in greater detail. Therefore, it should be understood that the scope of the present disclosure is not limited to the exemplary embodiments described above or by the use of such terms unless limited by the appended claims. Also, it should be apparent to those skilled in the art that various alterations, substitutions, and modifications may be made within the scope of the appended claims or equivalents thereof.

The present disclosure is thus not limited to the example embodiments described above, and rather intended to include the following appended claims, and all modifications, equivalents, and alternatives falling within the spirit and scope of the following claims.

### DESCRIPTION OF SYMBOLS

100: apparatus for analysing a signal
170: memory
171: signal processing module
172: clustering module
173: model generating module
174: artificial intelligence model
175: learning module

What is claimed is:

1. A method for analysing a signal, comprising:
collecting signals needed to learn signal analysis;
clustering features of the signals on a feature space;
generating a signal analysis model for each of clusters formed by the clustering; and
integrating outputs of the signal analysis models generated for the clusters.

2. The method according to claim 1,
wherein the collecting the signals comprises collecting biomedical signals generated from a testee to predict a state of the testee.

3. The method according to claim 2,
wherein the collecting the biomedical signals comprises,
under a current situation or environment of the testee:
receiving raw biomedical signals from the testee; and
processing the raw biomedical signals.

4. The method according to claim 2,
wherein the biomedical signals comprise at least one of a movement signal, a breathing signal, or a heartbeat signal of the testee.

5. The method according to claim 1,
wherein the clustering comprises:
extracting features from the signals that are in time series; and
clustering the features on the feature space.

6. The method according to claim 1,
wherein the clustering comprises determining probabilities for the clusters formed by the features extracted from the signals.

7. The method according to claim 1,
wherein the generating the signal analysis model comprises generating a signal analysis model that is adaptive to a situation or environment of a signal source by using an ensemble technique.

8. The method according to claim 1,
wherein the integrating the outputs of the signal analysis models comprises determining, based on probability values for the clusters formed by the features extracted from the signals, an ensemble weight to be applied to each of the clusters.

9. The method according to claim 8,
further comprising predicting a state of a signal source based on the result of integrating the outputs.

10. The method according to claim 9,
wherein the predicting the state of the signal source comprises predicting the state of the signal source by using the signal analysis model to which the ensemble weight is applied.

11. The method according to claim 8,
wherein the determining the ensemble weight comprises setting the ensemble weight as a linear or nonlinear function of the probability values for the clusters.

12. An apparatus for analysing a signal, comprising:
a processor configured to process collected signals, extract features from the signals, generate a signal analysis ensemble model to be trained via learning of the features, and control an output of the signal analysis ensemble model;
a learning processor configured to train the signal analysis ensemble model via learning; and
the signal analysis ensemble model configured to obtain a trained deep neural network assemble for predicting, via training, a state of a signal source from which the signals originated,
wherein the signal analysis ensemble model is configured to:
comprise a plurality of signal analysis models corresponding to clusters formed by clustering of the features, and
integrate and output outputs of the plurality of signal analysis models generated for the clusters.

13. The apparatus according to claim 12,
wherein the signals comprise biomedical signals generated from a testee as the signal source.

14. The apparatus according to claim 13,
wherein the processor is configured to process the biomedical signals collected under a current situation or environment of the testee.

15. The apparatus according to claim 12,
wherein the processor is configured to extract the features from the biomedical signals of the testee, and cluster the extracted features on a feature space by using a clustering model.

16. The apparatus according to claim 13,
wherein the processor is configured to determine probabilities for the clusters formed by the features extracted from the signals.

17. The apparatus according to claim 13,
wherein each of the plurality of signal analysis models corresponds to a biomedical signal analysis model that is adaptive to a situation or environment of the testee as the signal source by using an ensemble technique.

18. The apparatus according to claim 13,
wherein the processor is configured to determine, based on probability values for the cluster formed by the features extracted from the signals, an ensemble weight to be applied to each of the cluster.

19. The apparatus according to claim 18,
wherein the processor is configured to predict a state of the testee by using the signal analysis ensemble model to which the ensemble weight is applied.

20. The apparatus according to claim 18,
wherein the processor is configured to set the ensemble weight as a linear or nonlinear function of the probability values for the clusters.
